(19) United States
(12) Patent Application Publication
(10) Pub. No.: US 2010/0131023 A1
(43) Pub. Date: May 27, 2010

(54) IMPLANTABLE MEDICAL DEVICES COMPRISING CATHODIC ARC PRODUCED STRUCTURES

(76) Inventors: Benedict James Costello, Berkeley, CA (US); Marc Jensen, Los Gatos, CA (US); Mark Zdeblick, Portola Valley, CA (US); Jeremy Frank, San Francisco, CA (US); Vladimer Gelfandbein, Mountain View, CA (US); Lawrence Arne, Redwood City, CA (US)

Correspondence Address: Proteus Biomedical, Inc.;Bozicevic, Field & Francis LLP, 1900 University Avenue, Suite 200, East Palo Alto, CA, 94303, US

(21) Appl. No.: 12/305,894

(22) PCT Filed: Jun. 21, 2007
(86) PCT No.: PCT/US07/14509

§ 371 Date: Feb. 2, 2010

Related U.S. Application Data
(60) Provisional application No. 60/805,464, filed on Jun. 21, 2006.
(60) Provisional application No. 60/805,578, filed on Jun. 22, 2006.
(60) Provisional application No. 60/805,581, filed on Jun. 22, 2006.
(60) Provisional application No. 60/805,576, filed on Jun. 22, 2006.
(60) Provisional application No. 60/862,928, filed on Oct. 25, 2006.
(60) Provisional application No. 60/888,908, filed on Feb. 8, 2007.
(60) Provisional application No. 60/890,306, filed on Feb. 16, 2007.
(60) Provisional application No. 60/917,297, filed on May 10, 2007.

(51) Int. Cl. A61N 1/05; C23C 14/34

(52) U.S. Cl. 607/2; 607/116; 204/192.12; 204/192.15; 204/298.09

(57) ABSTRACT

Implantable medical devices that include cathodic arc produced structures are provided. Cathodic arc produced structures of the invention may be thick, stress-free metallic structures that have configurations heretofore not available in implantable medical devices. In yet other embodiments, the structures may be crenulated or porous layers. Also provided are methods of producing implantable medical devices as well as systems for practicing the subject methods.

### CROSS REFERENCE TO RELATED APPLICATIONS

Pursuant to 35 U.S.C. §119 (e), this application claims priority to the filing dates of: U.S. Provisional Application Ser. No. 60/805,464 titled “Implantable Medical Devices Comprising Cathodic Arc Produced Structures” and filed on Jun. 21, 2006; U.S. Provisional Application Ser. No. 60/805,578 titled “Cathodic Arc Deposition Hermetically Sealed Implantable Structures” and filed on Jun. 22, 2006; U.S. Provisional Application Ser. No. 60/805,576 titled “Implantable Medical Devices Comprising Cathodic Arc Produced Structures” and filed on Jun. 22, 2006; U.S. Provisional Application Ser. No. 60/805,581 titled “Noble Metal Compounds Produced by Cathodic Arc Deposition” and filed on Jun. 22, 2006; U.S. Provisional Application Ser. No. 60/862,928 titled “Medical Devices Comprising Cathodic Arc Produced Microstrip Antennas” and filed on Oct. 25, 2006; U.S. Provisional Application Ser. No. 60/888,908 titled “Metal Binary And Ternary Compounds Produced by Cathodic Arc Deposition” and filed on Feb. 8, 2007; U.S. Provisional Application Ser. No. 60/890,306 titled “Metal Binary And Ternary Compounds Produced by Cathodic Arc Deposition” and filed on Feb. 16, 2007; and U.S. Provisional Application Ser. No. 60/917,297 titled “Mental Binary And Ternary Compounds Produced by Cathodic Arc Deposition” and filed on May 10, 2007; the disclosures of which applications are herein incorporated by reference.

### INTRODUCTION

A variety of different kinds of implantable medical devices (IMD) are known in the art, which devices may have one or more different functions, including, but not limited to: monitoring of physiological parameters; delivery of pharmacological agents; and delivery of electrical stimuli, etc.

There is a continued desire in the field to produce increasingly complex implantable medical devices that have ever smaller dimensions, such that the capabilities of the device may be enhanced while the profile of the device may be reduced. To this end, a variety of different fabrication techniques have been employed to make implantable medical devices.

Published U.S. Patent application nos. 20060058588; 20050160827; 20050160826; 20050160825; 20050160824; 20050160823; 20040254483; 20040220637; 20040215049 and 20040193021 by some of the current inventors describe the use of planar processing techniques, such as Micro-Electro-Mechanical Systems (MEMS) fabrication, in the production of medical devices. Deposition techniques that may be employed in certain aspects of fabrication the structures include, but are not limited to: electroplating, plasma spray, sputtering, e-beam evaporation, physical vapor deposition, chemical vapor deposition, plasma enhanced chemical vapor deposition, etc. Material removal techniques include, but are not limited to: reactive ion etching, anisotropic chemical etching, isotropic chemical etching, planarization, e.g., via chemical mechanical polishing, laser ablation, electronic discharge machining (EDM), etc. Also of interest are lithographic protocols.

One known type of material deposition protocol is cathodic arc deposition. In cathodic arc plasma deposition, a form of ion beam deposition, an electrical arc is generated between a cathode and an anode that causes ions from the cathode to be liberated from the cathode and thereby produce an ion beam. The resultant ion beam, i.e., plasma of cathodic material ions, is then contacted with a surface of a substrate (i.e., material on which the structure is to be produced) to deposit a structure on the substrate surface that is made up of the cathodic material, and in certain embodiments element(s) obtained from the atmosphere in which the substrate is present. A number of patents and published applications are available which describe various cathodic arc deposition protocols and systems. Such publications include U.S. Pat. Nos. 6,929,727; 6,821,399; 6,770,178; 6,702,931; 6,663,755; 6,645,354; 6,608,432; 6,602,390; 6,548,817; 6,465,793; 6,465,780; 6,436,254; 6,409,898; 6,331,332; 6,319,369; 6,261,421; 6,224,726; 6,036,828; 6,031,239; 6,027,619; 6,026,763; 6,009,829; 5,972,185; 5,932,078; 5,902,462; 5,895,559; 5,518,597; 5,468,363; 5,401,543; 5,317,235; 5,282,944; 5,279,723; 5,269,896; 5,126,030; 4,936,960; and Published U.S. Application Nos.: 20050249983; 20050189218; 20050181238; 20040168637; 20040103845; 20040055538; 20040026242; 20030209424; 20020144893; 20020140334 and 20020139662.

While cathodic arc deposition protocols are known, to the knowledge of the inventors of the present application such protocols have, to date, been used solely in non-medical device applications, such as the production of coatings on large industrial elements, such as rotor blades, etc., as well as in the production of jewelry. To the best of the inventor's knowledge, cathodic arc deposition has not been employed in the production of medical devices and components thereof.

Despite the significant progress that has been made by applying planar processing protocols, such as MEMS protocols, in medical device design and fabrication, there continues to be a need for the development of new fabrication techniques that can be employed to fabricate implantable medical devices that have ever increasing complexity and ever decreasing size specifications. Of particular interest would be the identification of a protocol that could be employed to produce compositions of deposited materials in a desired form, e.g., thick, stress-free layers, porous layers, and layers having crenulations, in a variety of different configurations, including complex three-dimensional configurations. The present invention satisfies this, and other, needs.

### SUMMARY

The present invention allows, for the first time, the production of thick, stress-free metallic structures on a substrate, even within substrate locations having high aspect ratios. Furthermore, alternative embodiments of the present invention allow for the production of porous metallic structures and metallic layers displaying crenulations on a surface thereof. The subject invention may be employed to produce a variety of different structures for implantable medical devices, including layers and three-dimensional components, e.g., electrical connections, coating layers, sealing layers, etc., where designs for such structures may be more intricate than heretofore possible. As such, the present invention allows for the production of medical device components that have not before been possible, thereby providing for significant increases in medical device capability while decreasing the overall size of the device.

Embodiments of the invention include implantable medical devices that have one or more cathodic arc produced structures, i.e., structures produced using a cathodic arc deposition process. The structures may be thick, stress-free metallic structures, porous layers and layers displaying crenulations. Embodiments of the invention further include methods of producing structures for medical device implants using cathodic arc deposition processes, as well as cathodic arc deposition systems that are configured to practice the methods of the invention.

### BRIEF DESCRIPTION OF THE FIGURES

FIG. 1 provides a schematic depiction of a cathodic arc plasma source according to an embodiment of the invention.

FIG. 2A to 2D provides pictures of a platinum layer deposited by cathodic arc deposition according to an embodiment of the invention.

FIG. 3 provides a picture of a platinum layer deposited by cathodic arc deposition according to an embodiment of the invention, where the layer displays surface crenulations.

FIGS. 4A and 4B show different three-dimensional views of a hermetically sealed integrated circuit according to an embodiment of the invention.

FIG. 5 shows one embodiment of a battery having a porous cathode under-layer according to one embodiment of the invention.

FIGS. 6A and 6B show different cross-sectional views of assemblies with multiple hermetically sealed integrated circuits according to alternative embodiments of the invention, where cathodic arc produced conductive feedthroughs are present.

FIG. 7A shows a cross section of an IC chip where a cathodic arc produced thick metal structure forms an antenna to one side of the chip. FIG. 7B shows a cross section of an IC chip where a thick metal forms an antenna on one side of the chip.

FIG. 8A is a schematic top view illustration of a first embodiment of an RF patch antenna formed on the exterior surface of a conductive housing of an implantable medical device that functions as the ground plane layer; FIG. 8B is a schematic top view illustration of a second embodiment of an RF patch antenna formed on the exterior surface of a conductive housing of an implantable medical device functioning as the ground plane layer; FIG. 8C is a schematic side cross-section view of the RF telemetry antenna taken along lines 15-15 of FIGS. 8A and 8B; FIG. 8D is a schematic top view illustration of a third embodiment of an RF telemetry antenna formed on the exterior surface of a dielectric housing of an implantable medical device having a ground plane layer formed inside the housing; FIG. 8E is a schematic side cross-section view of the RF telemetry antenna taken along lines 17-17 of FIG. 8D; FIG. 8F is a schematic top view illustration of a fourth embodiment of an RF telemetry antenna having a radiator patch layer formed within the surface of an insulative, dielectric housing of an implantable medical device; and FIG. 8G is a schematic side cross-section view of the RF telemetry antenna taken along lines 19-19 of FIG. 8F.

FIG. 9A shows a cross section of an IC chip where a thick metal forms a multiplicity of electrodes attached to the chip. FIG. 9B shows a cross section of an IC chip where a thick metal forms a multiplicity of electrodes attached to the chip and those electrodes are formed into a shape.

FIG. 10 is a simplified schematic view of an implantable medical device and an external programmer employing the improved RF telemetry antenna of the present invention;

FIG. 11 is a simplified circuit block diagram of major functional uplink and downlink telemetry transmission functions of the external programmer and implantable medical device of FIG. 10;

FIG. 12 is a block diagram of a medical diagnostic and/or treatment platform according to an embodiment of the present invention;

FIG. 13 shows a patient with multiple remote devices implanted at various locations in his or her body according to an embodiment of the present invention;

### DETAILED DESCRIPTION

The present invention provides the medical device designer and manufacturer with an important new tool for producing medical device components. Using the protocols and systems of the invention, the medical device manufacturer can produce thick, stress-free metallic structures that heretofore could not be made. Furthermore, metallic stress-free structures having configurations that heretofore could not be produced are now possible. Additional structures that can be produced include porous layers and layers that exhibit crenulations on their surface. As such, the invention provides medical device designers with expanded capabilities in the design of medical device components, and enables the production of such new designs.

In further describing the invention in greater detail, embodiments of medical devices that include cathodic arc produced structures are reviewed first, followed by a review of cathodic arc deposition methods for fabricating the structures and systems configured for use in practicing the methods.

Implantable Medical Devices that Include Cathodic Arc Produced Structures

As summarized above, the invention provides implantable medical devices that include a cathodic arc produced structure(s). By implantable medical device is meant a device that is configured to be positioned on or in a living body, where in certain embodiments the implantable medical device is configured to be implanted in a living body. Embodiments of the implantable devices are configured to maintain functionality when present in a physiological environment, including a high salt, high humidity environment found inside of a body, for 2 or more days, such as about 1 week or longer, about 4 weeks or longer, about 6 months or longer, about 1 year or longer, e.g., about 5 years or longer. In certain embodiments, the implantable devices are configured to maintain functionality when implanted at a physiological site for a period ranging from about 1 to about 80 years or longer, such as from about 5 to about 70 years or longer, and including for a period ranging from about 10 to about 50 years or longer. The dimensions of the implantable medical devices of the invention may vary. However, because the implantable medical devices are implantable, the dimensions of certain embodiments of the devices are not so big such that the device cannot be positioned in an adult human. For example, the implantable medical devices may be dimensioned to fit within the vasculature of a human.

The function of the implantable medical devices of the invention may vary widely, including but not limited to: cardiac devices, drug delivery devices, analyte detection devices, nerve stimulation devices, etc. As such, implantable medical devices include, but are not limited to: implantable cardiac pacemakers, implantable cardioverter-defibrillators, pacemaker-cardioverter-defibrillators, drug delivery pumps, cardiomyostimulators, cardiac and other physiologic monitors, nerve and muscle stimulators, deep brain stimulators, cochlear implants, artificial hearts, etc. Illustrative embodiments of various types of implantable medical devices of the invention are reviewed in greater detail below.

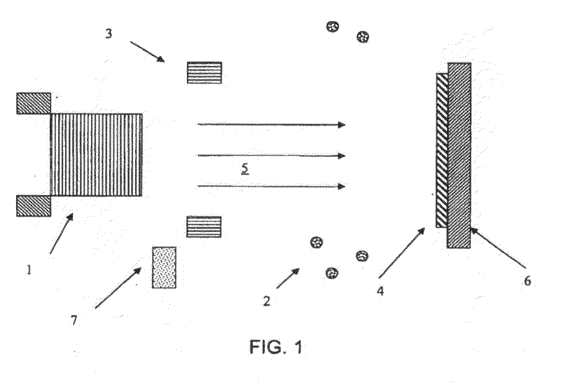

As summarized above, implantable medical devices of the invention include one or more structures that are produced by a cathodic arc plasma deposition process. An example of a cathodic arc plasma deposition system is shown in FIG. 1. In cathodic arc plasma deposition, a form of ion beam deposition, an electrical arc is generated between a cathode 1 and an anode 3 that causes ions from the cathode 1 to be liberated from the cathode and thereby produce an ion beam 5. The resultant ion beam, i.e., plasma of cathodic material ions, is then contacted with a surface of a substrate 6 (i.e., material on which the structure is to be produced) to deposit a structure 4 on the substrate surface that is made up of the cathodic material, and in certain embodiments element(s) obtained from the atmosphere in which the substrate is present. See e.g., FIG. 1. Where desired, e.g., where the product structure is a compound of the cathode material and one or more additional elements (such as carbon, nitrogen, etc.) a gas inlet 7 may be provided for introduction of a source gas for the one or more additional elements of interest. Also shown in FIG. 1 are neutral macroparticles 2, which particles may or may not be filtered from the plasma prior to deposition, as desired.

The cathodic arc produced structures of the invention are, in certain embodiments, thick, stress-free metallic structures. In certain embodiments, the structures range in thickness from about 0.01 μm to about 500 μm, such as from about 0.1 μm to about 150 μm. In certain embodiments, the structures have a thickness of about 1 μm or greater, such as a thickness of about 25 μm or greater, including a thickness of about 50 μm or greater, where the thickness may be as great at about 75, 85, 95 or 100 μm or greater. In certain embodiments, the thickness of the structures ranges from about 1 to about 200, such as from about 10 to about 100 μm.

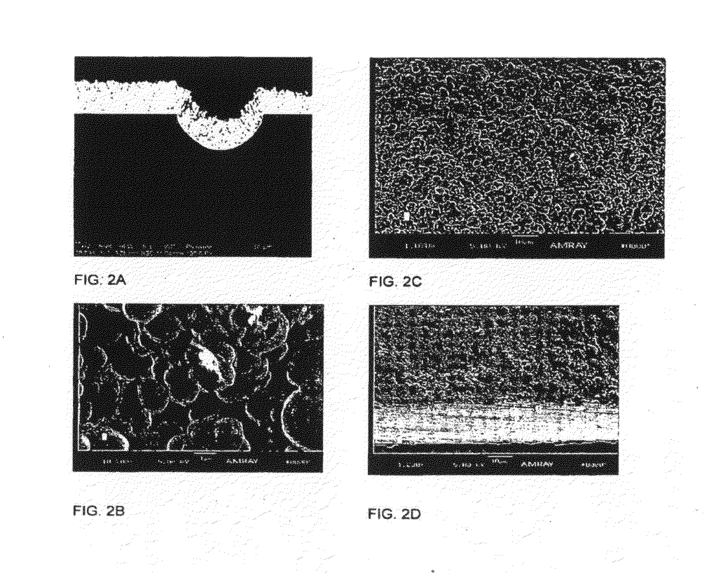

The cathodic arc produced structures are, in certain embodiments, stress-free. By “stress-free” is meant that the structures are free of defects that would impair the functionality of the structure. As such, “stress-free” means low stress as compared to stress that would case the structures to pull away, e.g., delaminate, from the substrate on which they are deposited. Accordingly, the structures are free of cracks, gaps, holes, or other defects, particularly those which would impair the function of the structure, e.g., the ability of the structure to seal an internal volume of the device, serves as a conductive element, etc. FIGS. 2A to 2D provide views of stress-free layers of platinum produced according to an embodiment of the invention.

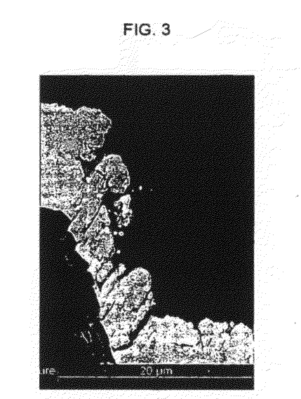

In yet other embodiments, the structure is a layer that exhibits surface crenulations. By surface crenulations is meant a series of projections separated by notches or crevices. The depth of a given notch as measured from the top of a given projection ranges, in certain embodiments, from about 0.1 μm to about 1000 μm, such as from about 1 μm to about 10 μm. FIG. 3 provides views of 10 μm thick layer of platinum exhibiting surface crenulations produced according to an embodiment of the invention. In yet other embodiments, the cathodic arc structures are porous structures.

As indicated above, the structures are, in certain embodiments, metallic structures. In certain embodiments, the metallic structures are structures that include a physiologically compatible metal, where physiologically compatible metals of interest include, but are not limited to: gold (au), silver (ag), nickel (ni), osmium (os), palladium (pd), platinum (pt), rhodium (rh), iridium (ir) titanium (ti), aluminum (al), vanadium (v), zirconium (zr), molybdenum (mo), iridium (ir), thallium (tl), tantalum (ta), and the like. In certain embodiments, the metallic structure is a pure metallic structure of a single metal. In yet other embodiments, the metallic structure may be an alloy of a metal and one or more additional elements, e.g., with the metals listed above or other metals, e.g., chromium (cr), tungsten (w), etc. In yet other embodiments, the structure may be a compound of a metal and additional elements, where compounds of interest include but are not limited to: carbides, oxides, nitrides, etc. Examples of compounds of interest include binary compounds, e.g., PtIr, PtTi, TiW and the like, as well as ternary compounds, e.g., carbonitrides, etc.

In certain embodiments, non-metallic structures are desired. For example, in certain embodiments the layer is carbon, such as diamond-like carbon. In these embodiments, the cathode material employed in the methods may be graphite. In certain embodiments, the diamond like carbon layer may be doped with one or more additional elements, e.g., nitrogen, gold, platinum, etc. Applications for such structures are varied, such as coatings for medical implants, etc.

In certain embodiments, the produced structure may include a gradient with respect to one element and the other, e.g., such as a metallic layer that has increasing amounts of a second element going from a first surface to a second surface. Additional materials that may make up a cathodic arc produced structure are described in copending PCT Application serial no. PCT/US2007/______ titled “Metal Binary and Ternary Compounds Produced by Cathodic Arc Deposition,” (having attorney docket no. PRTS-048WO2) and filed on Jun. 21, 2007, the disclosure of which is herein incorporated by reference.

The substrate on which the metallic structures are cathodic arc deposited may be made up of a variety of different materials and have a variety of different configurations. The surface of the substrate on which deposition occurs may be planar or non-planer, e.g., have a variety of holes, trenches, etc. The substrate may be made up of any of a number of different materials, such as silicon, (e.g., single crystal, polycrystalline, amorphous, etc), silicon dioxide (glass), ceramics, silicon carbide, alumina, aluminum oxide, aluminum nitride, boron nitride, beryllium oxide, among others; diamond-like carbon, sintered materials, etc. The substrate may be a composite of a conductive and semi-conductive materials (such as Ge), including highly doped and/or heated semi-conductor silicon, e.g., a circuit layer, such as those described below, where one or more conductive elements are present on a semi or non-conductive support.

The cathodic arc produced structures of the subject implantable medical devices may have a variety of different configurations and serve a variety of different functions in the implantable medical device in which they are found. For example, in certain embodiments the cathodic arc produced structures are layers that cover a least a portion of a surface of a component of the implantable medical device. In these embodiments, the layers may cover only a fraction of the surface or they may cover all of the surface, depending on the function of the layer. The layers may have a number of different purposes. In other embodiments, the cathodic arc produced structures are non-layer structures, e.g., feed throughs, identifiers, antennas, etc., which non-layer structures may also have a number of different functions. Representative layer and non-layer structures are now reviewed in greater detail.

### Structures Having a Layer Configuration

As summarized immediately above, in certain embodiments the cathodic arc produced structures are layer structures, by which is meant that they have a layer configuration, thereby having a length and width that is significantly greater than their height, e.g., by at 5-fold or more, such as by 50-fold or more and including by 100-fold or more. Depending on the purpose of the layer structure, the layer can have a variety of different configurations.

### Sealing Layers

In certain embodiments, the layer serves to seal an internal volume of the device from the external environment of the device, where such a sealing layer may be present on a single surface of the device or on more than one surface of the device, e.g., where the sealing layer may be present on every surface of the device. In certain embodiments, the cathodic arc deposited structures are the sealing layers described in PCT/US2005/046815 titled “Implantable Hermetically Sealed Structures” and published as WO 2006/069323; and PCT/US2007/09270 titled “Void-Free Implantable Hermetically Sealed Structures,” filed on Apr. 12, 2007; the disclosures of which are herein incorporated by reference. The layers may encapsulate the entire device, e.g., to provide a sealing layer that encloses the entire device, i.e., all surfaces of the device, or just a portion thereof, such as is described in PCT application serial no. PCT/US2007/09270 titled “Void-Free Implantable Hermetically Sealed Structures,” filed on Apr. 12, 2007; the disclosure of which is herein incorporated by reference.

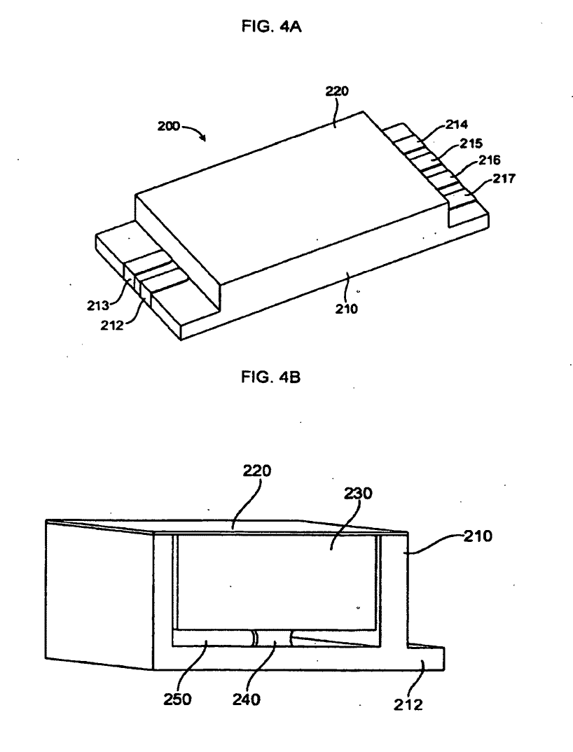

An example of an implantable medical device that includes a cathodic arc produced layer is provided in FIGS. 4A and 4B. FIG. 4A provides a three-dimensional view of a hermetically sealed structure according to an embodiment of the invention. In FIG. 4A, structure 200 includes holder 210 and sealing layer 220, where the sealing layer 220 has been deposited via cathodic arc deposition. Sealing layer 220 and holder 210 are configured to define a hermetically sealed volume (not shown) inside the holder. Also shown are external connector elements 212, 213, 214, 215, 216 and 217, which are coupled to conductive feedthroughs (not shown) present in the bottom of the holder.

FIG. 4B provides a three-dimensional cut-away view of a hermetically sealed structure according to an embodiment of the invention. In FIG. 4B holder 210 and sealing layer 220 define a hermetically sealed volume 250 what holds an effector (e.g., comprising an integrated circuit) 230. The effector 230 is electrically coupled to the conductive (e.g., platinum) feedthroughs or vias 212 with a solder alloy (e.g., lead tin, gold tin, silver tin, or other suitable alloys) 240.

In certain embodiments, any space between an effector and the walls of the holder and/or sealing layer may be occupied by an insulating material. Any convenient insulating material may be employed, where representative insulating materials include, but are not limited to: liquids, e.g., silicon oil, elastomers, thermoset resins, thermoset plastics, epoxies, silicones, liquid crystal polymers, polyamides, polyimides, benzo-cyclo-butene, ceramic pastes, etc.

Additional examples of sealing layers that may be produced according to embodiments of the invention are provided in published PCT application No. WO 2006/069323; and pending PCT application No. PCT/US2007/09270 titled “Void-Free Implantable Hermetically Sealed Structures,” filed on Apr. 12, 2007; the disclosures of which are herein incorporated by reference. the disclosure of which is herein incorporated by reference.

### Crenulated Layers

As summarized above, the cathodic arc deposited structures may be crenulated layers, in that they exhibit a crenulated surface, such as seen in FIG. 3. Such layers find use in a variety of different applications.

For example, providing a crenulated surface on an implant finds use in applications were osseointegration is desired. The crenulated layer can be produced in both deposited metals (e.g., Pt) and metallic compounds (e.g., TiO₂). The crenulated layers can be deposited on a variety of bone implant devices, where the implant devices may be metal implants or polymeric, e.g., PEEK and PEKK, implants. Bone implant devices of interest include, but are not limited to: hip implants, bone screws, dental implants, plates, support rods, etc.

Where desired, the crenulations can be filled with active agents, e.g., to aid bone growth and retard bacterial growth. Active agents of interest include, but are not limited to: organic polymers, e.g. proteins, including bone associated proteins which impart a number of properties, such as enhancing resorption, angiogenesis, cell entry and proliferation, mineralization, bone formation, growth of osteoclasts and/or osteoblasts, and the like, where specific proteins of interest include osteonectin, bone sialoproteins (Bsp), α-2HS-glycoproteins, bone Gla-protein (Bgp), matrix Gla-protein, bone phosphoglycoprotein, bone phosphoprotein, bone proteoglycan, protolipids, bone morphogenic protein, cartilage induction factor, platelet derived growth factor, skeletal growth factor, and the like; particulate extenders; inorganic water soluble salts, e.g. NaCl, calcium sulfate; sugars, e.g. sucrose, fructose and glucose; pharmaceutically active agents, e.g. antibiotics (such as gentamycin); and the like.

Crenulated layers are also of interest as active agent depots on devices other than bone implant devices. For example, active agent coated stents are of interest in certain medical applications. Such devices may include a crenulated layer of the invention in which the notches or crevices of the layer serve as depots or reservoirs for an active agent of interest, where the crenulations can be filled by saturating the surface with a drug in solution, e.g., under pressure. Active agents of interest include, but are not limited to: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) anti-neoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines, and (r) hormones. Of interest in certain embodiments are anti-inflammatory agents, e.g., glucocorticosteroids, such as dexamethasone, etc.

### Porous Layers

Also of interest are porous cathodic arc deposited layers. Porous cathodic arc deposited layers find use in a variety of different medical device components; such as but not limited to: electrodes, implant coatings, etc. One type of component of interest in which cathodic arc produced porous layers find use is high surface area electrode components, where such components find use in a variety of different implantable devices, e.g., as effectors (such as sensors or stimulators), as components of power sources, etc.

Embodiments of the inventive batteries of the present invention include structures having a high surface area cathode. By high surface area cathode is meant a cathode having a surface area that is about 2 fold or greater, such at about 10 fold or greater, than the area of the surface of a solid support that is covered by the cathode in the battery. In certain embodiments, the active area of the electrode has a surface area that is 10⁻³ or more, such as 10⁻⁷ or more and include 10⁻⁹ or more greater than the corresponding surface area resulting from the basic geometrical shape of the electrode. In certain embodiments, the surface area of the cathode ranges from about 0.01 mm² to about 100 mm², such as from about 0.1 mm² to about 50 mm² and including from about 1 mm² to about 10 mm². In certain embodiments, the high surface area cathode is obtained by having a cathode that is made up of an active cathode material present on a porous under-layer. In addition, the batteries include an anode present on a surface of a solid support.

Depending on the particular embodiment, the cathode and anode may be present on the same support or different supports, e.g., where two or more different supports are bonded together to produce the battery structure, e.g., as is present in a “flip-chip” embodiment. Similarly, the number of cathodes and anodes in a given battery may vary greatly depending on the embodiment, e.g., where a given embodiment may include a single battery having one anode and cathode, a single battery having multiple anodes and/or cathodes, or two or more distinct batteries each made up of one or more cathodes and/or anodes. Battery configurations of interest include, but are not limited to, those disclosed in application Ser. No. 60/889,870 titled “Pharma Informatics System Power Source Having High Surface Area Cathodes” and filed on Feb. 14, 2007; the disclosure of which is herein incorporated by reference.

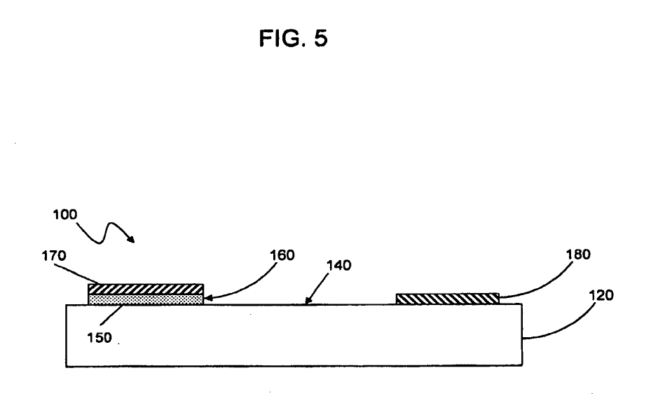

FIG. 5 provides a schematic illustration of battery according to an embodiment of the invention. The battery 100 shown in FIG. 5 includes a solid support 120 having an upper surface 140. Present on the upper surface 140 is cathode 160 and anode 180. Cathode 160 includes porous under-layer 150 and active cathode material 170. Each of these elements is now described in greater detail below. While the embodiment depicted is where the cathode includes a porous underlayer, in certain embodiments it is the anode that includes a porous underlay, while in yet other embodiments both a cathode and anode have the porous underlayer.

The porous under-layer 150 is a layer that mechanically supports the active cathode material 170 and provides for current passage between the cathode material and elements, e.g., circuitry, present on the solid support 120 (described in greater detail below). The porous under-layer may be fabricated from a variety of different materials, such as conductive materials, e.g., copper, titanium, aluminum, graphite, etc., where the materials may be pure materials or materials made up of two or more elements, e.g., as found in alloys, etc. The thickness of the under-layer may vary, where in certain embodiments the thickness ranges from about 0.01 to about 100 μm, such as from about 0.05 to about 50 μm and including from about 0.01 to about 10 μm. The dimensions of the porous under-layer with respect to length and width on the surface of the solid support may or may not be coextensive with the same dimensions of the active cathode material, as desired.

As summarized above, the cathode under-layer may be rough or porous. The porosity or roughness of the under-layer may vary, so long as it imparts the desired surface area to the cathode. In certain embodiments, the porosity or roughness of the cathode under-layer is chosen to provide an effective surface area enhancement of about 1.5 times or more to about 1000 times or more, e.g., from about 2 to about 100 time or more, such as from about 2 to about 10 times or more, greater than that obtained from a comparable cathode that lacks the porous underlayer. Surface area enhancement can be determined by comparing the electrochemical capacitance or cyclic voltammogram of the rough or porous electrode with that of a smooth electrode of the same material. Roughness may also be determined by other techniques, such as atomic force microscopy (AFM), electron microscopy, or Brunauer-Emmett-Teller (BET) analysis.

According to the invention, a cathodic arc deposition protocol is employed to produce the desired porous cathode under-layer. In such protocols, a cathodic arc generated metallic ion plasma is contacted with a surface of a substrate, e.g., 120, under conditions sufficient to produce the desired structure of the porous cathode under-layer, e.g., as described above. The cathodic arc generated ion plasma beam of metallic ions may be generated using any convenient protocol. As detailed below, in generating an ion beam by cathodic arc protocols, an electrical arc of sufficient power is produced between a cathode and one or more anodes so that an ion beam of cathode material ions is produced. The resultant beam is directed to at least one surface of a substrate in a manner such that the ions contact the substrate surface and produce a structure on the substrate surface that includes the cathode material.

Present on top of the porous cathode (or anode) under-layer is the active cathode (or anode) material. The active cathode material may comprise a variety of different materials. In certain embodiments, the cathode material includes copper, where of particular interest in certain embodiments are cuprous iodide (CuI) or cuprous chloride as the cathode material. Where desired, e.g., to enhance voltage of the battery, the active material may be doped with additional elements, e.g., sulfur, etc. The active cathode material may be provided onto the porous under-layer using any convenient protocol, including such as electrodeposition, e.g., electroplating, or evaporation, e.g., chemical vapor deposition. The anode material may comprise a variety of different materials. In certain embodiments, the anode material includes magnesium (Mg) metal or magnesium alloy. The active anode material may be provided onto the porous under-layer using any convenient protocol, such as electrodeposition, e.g., electroplating, or evaporation, e.g., chemical vapor deposition.

### Structures Having a Non-Layer Configuration

In certain embodiments, the cathodic arc deposited structure is a non-layer, three-dimensional component of the medical device, where such components may vary widely in terms of configuration and function. Three-dimensional components of interest that may be produced using the subject deposition protocols, described in greater detail below, include but are not limited to: conductive elements, e.g., vias or other conductive lines found in an implantable medical device; communication elements, e.g., antennae; identification components, e.g., identification markings on the device; orientation components, e.g., surface elements that are employed to orient the device under imaging; effectors, such as tissue interaction elements, e.g., electrodes, etc.

### Vias and Analogous Structures

In certain embodiments, the cathodic arc deposited structure is a three-dimensional conductive element of the device. In certain embodiments, the conductive element serves to conductively connect two distinct structures of the device. In certain embodiments, the conductive element is a via, where the via may be present in a high aspect ratio passage of the device. By high aspect ratio passage is meant a passage having a height to width ratio of up to about 100 or higher, such as from about 1 to about 50.

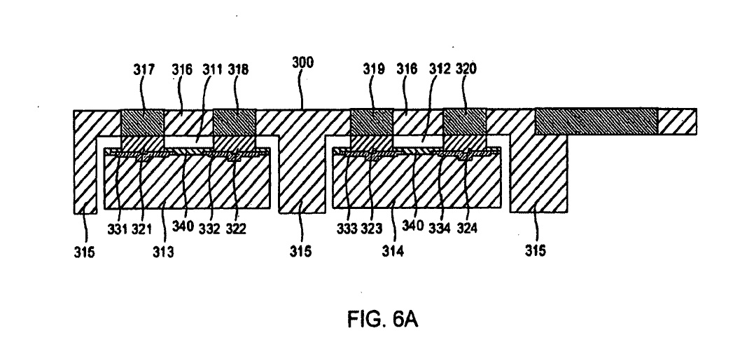

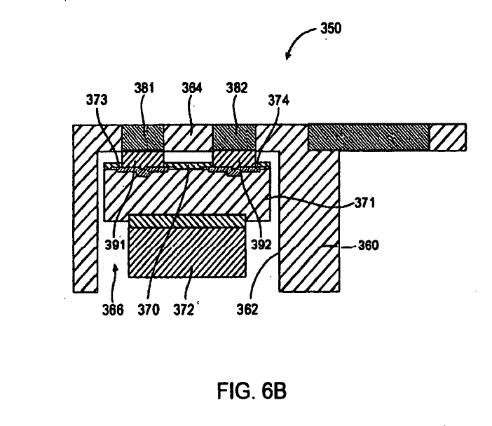

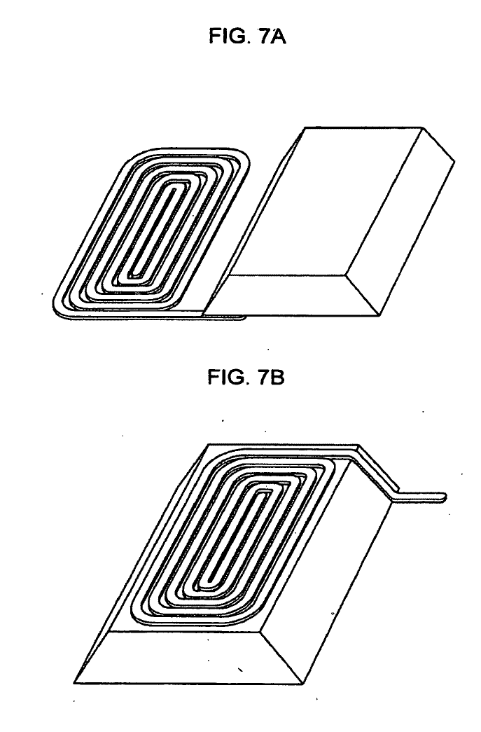

FIG. 6A provides a cross-sectional view of a hermetically sealed structure that includes cathodic arc produced conductive feedthroughs according to an embodiment of the invention. In this embodiment, the holder 300 includes two distinct wells 311 and 312, positioned side by side, e.g., in an array format, where each well houses two different effectors 313 and 314 (e.g., integrated circuits). Each well includes sides 315 and a bottom 316. Also shown in the bottom of each well are cathodic arc produced conductive feedthroughs 317, 318, 319 and 320. Electrically coupling the traces 331, 332, 333 and 334 of integrated circuits 313 and 314 to the conductive feedthroughs are solder connections 321, 322, 323 and 324. Separating the different solder connections from each other is insulating material 340. Although not shown, a suitable insulating material may also be present in the spaces between the effectors and the sides/bottom of the wells of the holder. In addition, a sealing layer is present on the surface opposite the feedthroughs, although not shown in FIG. 6A. While the depiction of FIG. 6A shows only two different integrated circuits hermetically sealed, structures of the invention may include many more integrated circuits, e.g., 4, 5, 6, or more circuits, in any convenient arrangement. One embodiment of the multiple chips per package design is to have a chip that is fabricated or otherwise designed to withstand higher voltages in one section of the assembly. The companion chip has a lower voltage tolerance than the first chip, but would not need the capacity of sustaining high voltages from cardiac pacing or other component demands from another part of the assembly. Both of those chips are dropped into the same hermetic packaging, e.g., in the same well or side by side wells, attached with a soldering process and then secured in place with an insulating material (i.e., potted), planarized or lapped back, e.g., as reviewed below, and then covered with a sealing layer.

While the above example provides guidance on synergistically providing two chips within a single inventive corrosion resistant hermetic package, these assemblies can handle up to 4, 5, 6, or more chips in a single assembly. In such larger scale assemblies, there is also the advantage that these assemblies can stacked on top of each other to add more functionality to the medical device components to be hermetically protected.

In FIG. 6B, structure 350 includes holder 360 with sides 362 and bottom 364 defining well 366. Present in well 366 are two different effectors 371 and 372 stacked on top of each other. Also shown in the bottom of each well are cathodic arc produced conductive feedthroughs 381 and 382. Electrically coupling the traces 373 and 374 of integrated circuit 371 to the conductive feedthroughs are solder connections 391 and 392. Separating the different solders from each other is insulating material 370. Although not shown, a suitable insulating material may also be present in the spaces between the effectors and the sides/bottom of the well of the holder. In addition, a sealing layer is present on the surface opposite the feedthroughs, although not shown in FIG. 6B.

### Communication Elements

As reviewed above, cathodic arc produced structures of interest include antenna structures. Because of the nature of the cathodic arc deposition process, antenna structures that heretofore could not be realized are now readily producible. Antenna structures may be straight or non-straight, e.g., curved, and have two dimension or three-dimensional configurations, as desired.

One embodiment of a non-straight antenna according to an embodiment of the invention that is readily produced via cathodic arc deposition protocols is shown in FIGS. 7A and 7B. FIG. 7A shows a cross section of an IC chip where a cathodic arc deposited thick metal structure forms an antenna to one side of the chip. The thick metal is free standing. The thick metal can also be supported by a substrate. FIG. 7B shows a cross section of an IC chip where a thick metal forms an antenna on one or more sides of the chip. The thick metal antenna depicted in these figures is readily produced via cathodic arc using an appropriate mask and depositing the antenna structure on a support through the mask.

In yet other embodiments, implantable medical devices of the invention include one or more microstrip patch antennas that are produced by a cathodic arc plasma deposition process. The microstrip patch antennas of the invention include an electrically conductive radiator patch layer present on a surface of dielectric substrate. In certain embodiments, a conductive ground plane layer is also present, e.g., on a surface of the dielectric substrate opposite the radiator conductive layer. In a medical device, the radiator patch layer may be coupled to transceiver circuitry of the medical device by a feedthrough extending through the dielectric substrate layer and the ground plane layer. An aspect of the invention is that the radiator patch layer is fabricated using a cathodic arc deposition process, in which the patch layer is deposited onto a surface of a dielectric substrate using cathodic arc plasma deposition protocols.

The cathodic arc produced conductive radiator patch layers of the microstrip patch antennas of the invention are, in certain embodiments, thick, stress-free metallic structures, e.g., as described above. While the physical dimensions of the patch layer may vary depending on the particular device configuration and use of the antenna, in certain embodiments the dimensions are chosen such that the antenna has an operating frequency ranging from about 200 to about 800 MHz, such as from about 300 to about 600 MHz and including from about 350 to about 450 MHz. Of interest in certain embodiments are antennas having an operating frequency ranging from about 400 to about 425 MHz, such as from about 400 to about 410 MHz, e.g., from about 402 to about 405 MHz. In certain embodiments, the patch layers range in thickness from about 0.01 μm to about 500 μm, such as from about 0.1 μM to about 150 μm. In certain embodiments, the structures have a thickness of about 1 μm or greater, such as a thickness of about 25 μm or greater, including a thickness of about 50 μm or greater, where the thickness may be as great at about 75, 85, 95 or 100 μm or greater. In certain embodiments, the thickness of the structures ranges from about 1 to about 200, such as from about 10 to about 100 μm. In certain embodiments, the patch layers have a surface area that ranges from about 1 cm² to about 10 cm², such as from about 1 cm² to about 4 cm². In certain embodiments, the patch layers have a longest dimension (e.g., diameter) ranging from about 1 cm to about 10 cm, such as from about 1 cm to about 6 cm.

As indicated above, the microstrip patch antenna structures include a radiator patch layer, where the patch layer is, in certain embodiments, a metallic layer. In certain embodiments, the metallic structures are structures that include a physiologically compatible metal, where physiologically compatible metals of interest include, but are not limited to: gold (Au), silver (Ag), nickel (Ni), Osmium (Os), palladium (Pd), platinum (Pt), rhodium (Rh), iridium (Ir) titanium (Ti), aluminum (Al), vanadium (V), zirconium (Zr), molybdenum (Mo), iridium (Ir), thallium (Tl), tantalum (Ta), and the like. In certain embodiments, the metallic structure is a pure metallic structure of a single metal. In yet other embodiments, the metallic structure may be an alloy of a metal and one or more additional elements, e.g., with the metals listed above or other metals, e.g., chromium (Cr), tungsten (W), etc. In yet other embodiments, the structure may be a compound of a metal and additional elements, where compounds of interest include but are not limited to: carbides, oxides, nitrides, etc. Of particular interest in certain embodiments are layers that include platinum, where such layers may be pure platinum or a combination of platinum and another element. Examples of compounds of interest include binary compounds; e.g., PtIr, PtTi, TiW and the like, as well as ternary compounds, e.g., carbonitrides, etc.

The substrate on which the metallic structures are cathodic arc deposited may be made up of a variety of different materials and have a variety of different configurations. The surface of the substrate on which deposition occurs may be planar or non-planer, e.g., have a variety of holes, trenches, etc. For example, holes in the substrate may surface as feedthroughs following deposition of the patch layer, as described above, and further elaborated in pending U.S. Provisional Application Ser. No. 60/805,576 filed on Jun. 22; 2006, the disclosure of which is hereby incorporated by reference. The substrate may be made up of any of a number of different materials, where dielectric materials are of interest, such as, but not limited to: silicon, (e.g., single crystal, polycrystalline, amorphous, etc), silicon dioxide (glass), ceramics, Teflon, etc.

In addition to the patch layer and the substrate, the subject microstrip antennas may also include a ground plane layer. The ground plane layer may be fabricated of any suitable conductive material and, in certain embodiments, may be part of the device with which the antenna is operatively coupled, e.g., the conductive housing of an implantable medical device.

In certain embodiments, the patch layer may also be covered with a protective layer, e.g., that is fabricated from a suitable dielectric material, which serves to protect the patch layer from body fluids. In certain embodiments, this protective layer may be configured as a radome structure, e.g., as described in U.S. Pat. No. 5,861,019, the disclosure of which is herein incorporated by reference.

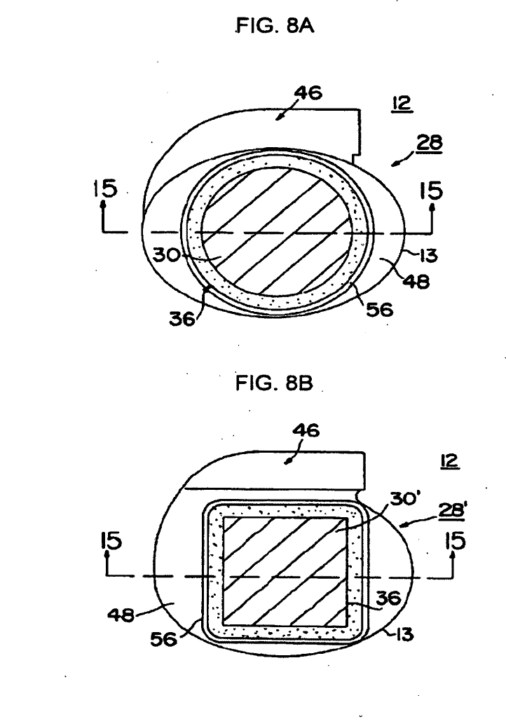

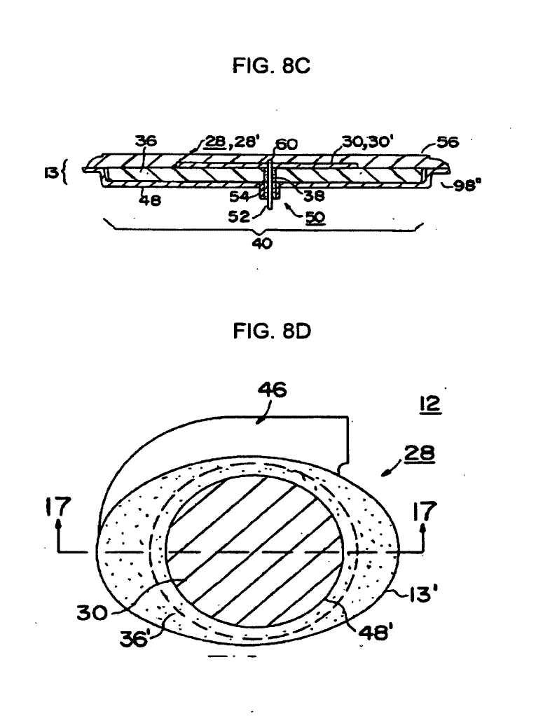

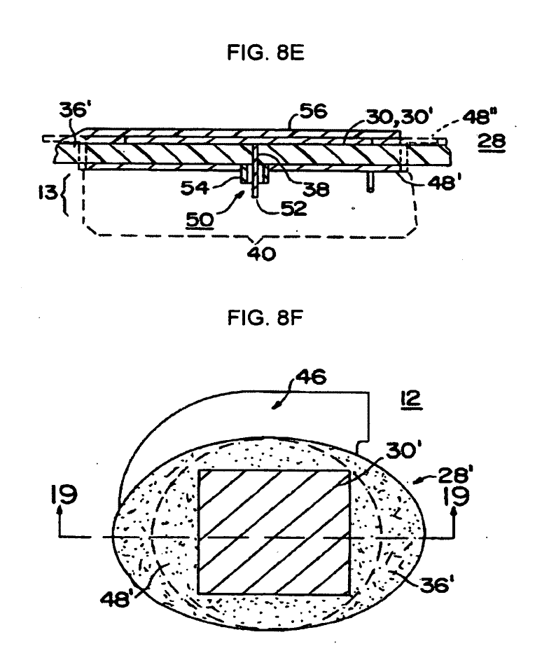

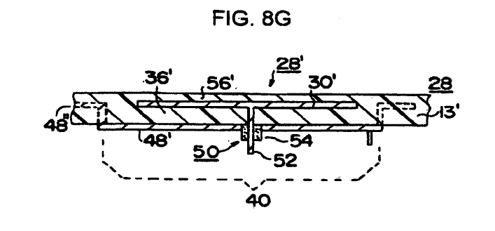

FIGS. 8A to 8C depict first and second embodiments of RF telemetry antennas 28, 28′ employing round and square (or rectangular) RF patch antenna plates or layers 30 and 30′, respectively, formed over a dielectric substrate layer 36 and ground plane layer 48. The ground plane layer 48 is part of the conductive housing 13 of an implantable pulse generator (IPG) device 12. The feedthrough pin 52 of feedthrough 50 extends through the ferrule 54 attached to the ground plane layer 48 and through the aligned hole 38 in the dielectric substrate layer 36 and the hole 60 in the radiator patch layer 30, 30′. The end of the feedthrough pin 52 is attached to the hole 60 by welding or the like. The actual location of the aligned holes 38 and 60 and the feedthrough 50 may be selected in the design phase to provide the best impedance match between the RF telemetry antenna 28, 28′ and the associated IPG transceiver.

The areas of the radiator patch layer 30, 30′ and the parallel ground plane layer 48 contribute to the RF frequency of the IPG RF telemetry antenna. In certain embodiments, the ground plane layer 48 area exceeds that of the radiator patch layer 30, 30′ Where it is necessary to size the radiator patch layer 30, 30′ and the underlying dielectric layer 36 to cover most of the major flat exterior surface of the IPG housing 13, then performance of the IPG RF microstrip antenna is compromised. In this case, the exterior housing 13 is preferably recessed in a circular housing recess 40 having a recess depth to accommodate the thickness of the dielectric substrate layer 36 and a recess diameter or length and width to accommodate the radiator patch layer 30, 30′. The housing recess 40 of the ground plane layer 48 provides an outward ground plane extension layer 48″ that is substantially co-planar with the radiator patch layer 30, 30′ that effectively increases the area of the microstrip antenna ground plane 48.

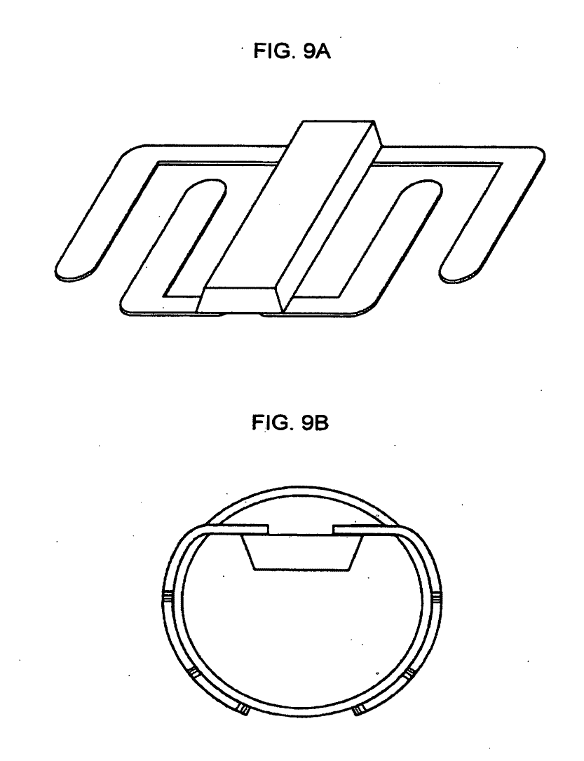

In order to improve the IPG RF telemetry antenna performance within the body fluids and tissue, it is desirable to employ a dielectric radome layer over the otherwise exposed surface of the radiator patch layer 30, 30′ that functions as a radome. Such an exemplary radome layer 56 is depicted in FIG. 9 and may be formed of the dielectric materials listed above. The radome layer 56 extends over the exterior surfaces of the radiator patch layer 30, 30′, the dielectric layer 36 and the outwardly extending edge region 48″ surrounding the housing recess 40 a suitable distance to the curved minor edge surface of the implantable medical device housing 13.

In the first and second embodiments, the conductive housing 13 and ground plane layer 48 are formed of a bio-compatible metal, e.g. titanium. When the implantable medical device is a unipolar IPG, an exposed surface portion of the housing 13 is used as an indifferent plate electrode for other electrical sensing and stimulation functions. The exposed indifferent electrode surface may be on the major, relatively flat, side of the IPG housing 13 opposite to the side where the RF telemetry antenna 28 is disposed. Disposing the RF telemetry antenna 28 to face toward the skin surface is advantageous for telemetry efficiency, and disposing the indifferent electrode surface inward is advantageous for both sensing electrical signals and electrical stimulation efficiency. As is known in the art, RF uplink and downlink telemetry transmissions can be synchronized with the operations of the implantable medical device to avoid times when the device operations involve electrical stimulation and/or sensing, although it may not be necessary to do so in the practice of the present invention.

FIGS. 8D and 8E depict a third embodiment of an RF telemetry antenna 28 with the radiator patch layer 30, 30′ formed on the exterior surface of a dielectric, ceramic, housing 13′ of an IPG 12 and having a ground plane layer 48′ formed as a conductive layer on the interior surface of the IPG housing 13′. Therefore, in this embodiment, the dielectric IPG housing 13′ constitutes and provides the dielectric substrate layer 36′ disposed between the ground plane layer 48′ and the radiator patch layer 30, 30′. It will be understood that the ground plane layer 48′ is insulated electrically from interior circuit components within the IPG housing. This embodiment also illustrates an alternative form of the feedthrough pin 52 which fills the dielectric layer hole 38 and is abutted against the interior surface of the radiator patch layer 30, 30′. In this case, the radiator patch layer 30, 30′ is optimally formed by thick or thin film deposition or adherence of a metal layer over the exterior surface of the dielectric IPG housing. The radiator patch layer 30, 30′ may be formed to extend into the hole 38 to the extent necessary to fill it and make secure electrical contact with the end of the feedthrough pin 52.

In this embodiment, if the ground plane layer 48′ is not large enough in area relative to the radiator patch layer 35′ then it may be necessary to form a rim or ring shaped, conductive, ground plane extension layer 48′ (shown in broken lines) extending around and spaced apart from the periphery of the radiator patch layer 30, 30′. The ground plane extension layer 48″ is electrically connected to the ground plane layer 48′ at least at one electrical connection, e.g., one or more plated through hole through the dielectric layer 36′. This electrical connection may alternatively be effected by providing the ground plane layer 48 as a single, dish shaped, layer that is fabricated with the major side of the medical device non-conductive housing 13′ to mimic the arrangement of the embodiment of FIGS. 8A to 8C.

In either variation, a radome layer 56 may also be formed overlying the exterior surfaces of the radiator patch layer 30, 30′, the dielectric layer 36′, and at least a portion of the ring shaped ground plane extension layer 48″ (if present) employing one of the above-identified materials.

FIGS. 8F and 8G depict a fourth embodiment of an RF telemetry antenna 28 having the radiator patch layer 30, 30′ formed as a layer within the insulative dielectric IPG housing 13′. In this embodiment, the outer layer of the non-conductive housing 13 functions as the radome layer 56′. The ground plane layer 48′ is formed as a conductive layer on the interior surface or within the IPG housing 13′ in the manner described above. The ground plane extension layer 48″ (shown in broken lines) is also formed as a layer that is substantially co-planar with the radiator patch layer 30, 30′ within the insulative dielectric IPG housing 13′ and is electrically connected with the ground plane layer 48 as described above.

The implantable pulse generators with which the subject antennas find use may vary in configuration. However, such devices typically include a power source and an electrical stimulation control element, which elements are present in a housing, e.g., that provides for a hermetically sealed structure of the contents inside the housing. Electrically coupled to the device, e.g., via an IS-1 interface, may be one or more cardiovascular leads (i.e., elongated structures) which have one or more electrodes positioned along their length. In certain embodiment, the lead is a multi-electrode (i.e., multiplex) lead which has two or more, such as four or more, 8 or more, 12 or more, 16 or more, 20 or more, 30 or more, 50 or more, electrodes positioned along its length. The lead may include one or more conductive members, e.g., wires, to provide for electrically coupling of the distal electrodes to the control element present in the IPG. As such, the lead may be a one wire lead, two wire lead or include more than two wires. However, in certain embodiments, the number of conductive elements, e.g., leads, is less than the number of electrodes on the lead. Of interest in certain embodiments are multi-electrode leads in which each electrode on the lead is individually addressable. Such includes include, but are not limited to, those described in Published PCT Application No. WO 2004/052182 and U.S. patent application Ser. No. 10/734,490, the disclosure of which is herein incorporated by reference. In certain embodiments, the electrodes present on the lead are segmented, e.g., to provide better current distribution in the tissue/organ to be stimulated. In such embodiments, the segmented electrodes are able to pace and sense independently with the use of a integrated circuit (IC) in the lead, such as a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. The IC allows each electrode to be addressed individually, such that each may be activated individually, or in combinations with other electrodes on the medical device. In addition, they can be used to pace in new and novel combinations with the aid of the multiplexing circuits on the IC. Of interest are segmented electrodes having quadrant electrode configuration, in which four segmented electrodes are configured as a band around the lead. The lead may include one or more of such bands, e.g., 2 or more, 3 or more, 4 or more, 5 or more, etc. Segmented electrode structures of interest include those described in PCT Application No. PCT/US2005/046811 filed on Dec. 22, 2005 and pending U.S. Provisional Application Ser. Nos. 60/793,295 filed Apr. 18, 2006 and 60/807,289 filed Jul. 13, 2006; the disclosures of which are herein incorporated by reference. In certain embodiments, the IC that is included with each segmented electrode structure is a hermetically sealed IC, e.g., as described in PCT Application No. PCT/US2005/046815 filed on Dec. 22, 2005 and pending U.S. Provisional Application Ser. Nos. 60/791,244 filed on Apr. 12, 2006 and 60/805,578 filed Jun. 22, 2006; the disclosures of which are herein incorporated by reference.

While the embodiments depicted in FIGS. 8A to 8G are IPGs, the subject antennas may be employed with any of a variety of different types of medical devices. As reviewed above, such devices include, but are not limited to: cardiac devices, drug delivery devices, analyte detection devices, nerve stimulation devices, etc. As such, implantable medical devices with which the subject antennas may be employed include, but are not limited to: implantable cardiac pacemakers, implantable cardioverter-defibrillators, pacemaker-cardioverter-defibrillators, pharmaceutical administration devices, e.g., implantable drug delivery pumps, cardiomyostimulators, cardiac and other physiologic monitors, nerve and muscle stimulators, deep brain stimulators, cochlear implants, artificial hearts, etc.

Further description regarding microstrip antennae of the present invention may be found in U.S. Provisional Application Ser. No. 60/862,928 titled “Medical Devices Comprising Cathodic Arc Produced Microstrip Antennas,” and filed on Oct. 25, 2006, the disclosure of which is herein incorporated by reference.

### Effectors

In certain embodiments, the cathodic arc deposited structure is a component of an effector of an implantable medical device. The term “effector” is generally used herein to refer to sensors, activators, sensor/activators, actuators (e.g., electromechanical or electrical actuators) or any other device that may be used to perform a desired function. In some embodiments, for example, effectors include a transducer and a processor (e.g., in the form of an integrated circuit (digital or analog). As such, embodiments of the invention include ones where the effector comprises an integrated circuit. The term “integrated circuit” (IC) is used herein to refer to a tiny complex of electronic components and their connections that is produced in or on a small slice of material, i.e., chip, such as a silicon chip. In certain embodiments, the IC is an IC as described in PCT Patent Application Serial No. PCT/US2005/031559 titled “Methods And Apparatus For Tissue Activation And Monitoring” filed on Sep. 1, 2005, the disclosure of which is herein incorporated by reference.

The effectors may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. patent application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/US05/39535 titled “Cardiac Motion Characterization by Strain Gauge”. These applications are incorporated in their entirety by reference herein.

An example of effectors that may be produced according to embodiments of the invention are electrodes. FIG. 9A shows a view of an IC chip where a thick metal forms a multiplicity of electrodes attached to the chip. The electrodes can be free standing or they can be supported by a substrate. The electrodes can be a capacitive in addition to being electrolytic electrodes. FIG. 9B shows a cross section of an IC chip where those electrodes are formed into a shape.

### Identifier Components

As summarized above, cathodic arc structures of interest also include medical device identifiers and/or orientation elements. For example, cathodic arc produced identifiers, e.g., words, symbols, bar codes, etc., fabricated from a radioopaque material may be provided on an implantable medical device. Following implantation of the device, identifying information about the device may be readily obtained without open surgery by imaging the device and obtaining the identifying information from the identifier. The identifier may be in the form of a words, symbols, a bar code, etc., where the identifier may provide various types of implant information, e.g., type of device, manufacturer of the device, serial no. of the device for unique identification of the device, etc. By cross referencing the identifier provided information with a database, further information may be readily obtained from a suitable database, such as when the device was implanted, who implanted the device, etc. All this information may be obtained without actually directly accessing the device through open surgery, but instead just by imaging the device with a suitable non-invasive imaging protocol.

In addition, the cathodic arc elements of interest include orientation elements, e.g., radioopaque bands, where such element can assist in proper placement of a device during implantation. For example, a non-radioopaque stent may be modified to include cathodic arc produced orientation elements on its outer surface, where such elements assist in placement of the stent during implantation.

### Methods

Also provided are methods of manufacturing implantable medical devices that include cathodic arc produced structures, where the methods include production of a structure using a cathodic arc deposition protocol.

The methods of the invention include contacting a cathodic arc generated metallic ion plasma with a surface of a substrate under conditions sufficient to produce the desired structure of the implantable medical device, e.g., as described above. The cathodic arc generated ion plasma beam of metallic ions may be generated using any convenient protocol. In generating an ion beam by cathodic arc protocols, an electrical arc of sufficient power is produced between a cathode and one or more anodes so that an ion beam of cathode material ions is produced. The resultant beam is directed to at least one surface of a substrate in a manner such that the ions contact the substrate surface and produce a structure on the substrate surface that includes the cathode material. See e.g., FIG. 1. Any convenient protocol for producing a structure via cathodic arc deposition may be employed, where protocols known in the art which may be adapted for use in the present invention include, but are not limited to those described in U.S. Pat. Nos. 6,929,727; 6,821,399; 6,770,178; 6,702,931; 6,663,755; 6,645,354; 6,608,432; 6,602,390; 6,548,817; 6,465,793; 6,465,780; 6,436,254; 6,409,898; 6,331,332; 6,319,369; 6,261,421; 6,224,726; 6,036,828; 6,031,239; 6,027,619; 6,026,763; 6,009,829; 5,972,185; 5,932,078; 5,902,462; 5,895,559; 5,518,597; 5,468,363; 5,401,543; 5,317,235; 5,282,944; 5,279,723; 5,269,896; 5,126,030; 4,936,960; and Published U.S. Application Nos.: 20050249983; 20050189218; 20050181238; 20040168637; 20040103845; 20040055538; 20040026242; 20030209424; 20020144893; 20020140334 and 20020139662; the disclosures of which are herein incorporated by reference. In certain embodiments, all of the surfaces of a substrate may be contacted with the plasma, e.g., to encapsulate the substrate (medical device) in a layer of cathodic arc deposited material, e.g., as described in PCT Application Serial No. PCT/2007/09270 filed on Apr. 12, 2007 titled “Void-Free Implantable Hermetically Sealed Structures”; the disclosure of which is herein incorporated by reference.

In certain embodiments, the cathodic arc deposition protocol employed is one that produces a thick, stress-free metallic structure on a surface of a substrate, e.g., as described above. As such, the method is one that produces a defect free metallic layer on a surface of the substrate that has a thickness of about 1 μm or greater, such as a thickness of about 25 μm or greater, including a thickness of about 50 μm or greater, where the thickness may be as great at about 75, 85, 95 or 100 μm or greater.

In accordance with the present invention, there is provided an improved methodology for depositing a layer of material on a substrate surface by cathodic arc deposition on a substrate surface. In certain embodiments, the substrate is subjected to deformation or force to produce layers of significantly improved character, relative to corresponding layers produced by deposition on a substrate not subjected to such deformation or force.

The method of stress engineering in accordance with the invention is also usefully employed in a wide variety of materials fabrication applications, such as for example, the formation on a silicon substrate of a cathodic arc or sputtered metal film whose growth stress is large and compressive. Since the coefficient of thermal expansion of the metal film is greater than that of the Si substrate material, the stress in the film at room temperature can be reduced by depositing at an elevated temperature. At the elevated deposition temperature, the film is still in compression, but as it cools on the substrate, it approaches a stress-free state. However, such elevated temperature film-formation conditions may be detrimental to other layers of an integrated circuit (IC) device present on the substrate. The same near-stress-free state can be obtained in accordance with the present invention by constraining the substrate during the sputter deposition, e.g., with a suitable constraining element, and then releasing the constraint after deposition, so that the top surface of the substrate is given the amount of compressive strain as is needed to be released from the sputtered metal layer.

The methodology of the invention is also applicable in the converse to the production of layers that have little growth stress, but must be deposited at a high temperature because of the constraints of a deposition or other elevated temperature process. In such case, the thermal expansion mismatch strain can be compensated in the practice of the invention by heating the substrate at the deposition temperature. In this way, there is little or no stress during deposition, and a stress is created during cooling, but the stress is then relieved by removing the wafer constraint.

In certain embodiments, contact of the plasma and the substrate surface in the subject methods occurs in a manner such that compressive and tensile forces experienced by deposited metal structure substantially cancel each other out so that the deposited metal structure is stress-free. In these embodiments, various parameters of the deposition process, including distance between the substrate and the cathode, temperature of the substrate and the power employed to produce the plasma are selected so that the product metallic layer is stress-free. In these embodiments, the distance between the substrate and the cathode may range from about 1 mm to about 0.5 m. The power employed to generate the plasma may range from about 1 watt to about 1 Killowatt or more, e.g., about 5 Killowatts or more.

In certain embodiments, the plasma beam is contacted with the substrate surface in a direction that is substantially orthogonal to the plane of the substrate surface on which the structures are to be produced. By “substantially orthogonal” is meant that the angle of the ion beam flow as it contacts the plane of the substrate ±15°, such as ±10°, including ±5° of orthogonal, including orthogonal, such that in certain embodiments the ion beam flow is normal to the plane of the substrate surface.

As such, embodiments of the methods include methods for deposition of stress-free films or layers utilized in medical implants wherein the properties of the layer materials are stress-dependent, by applying heating or cooling to the substrate (or compressive force) of choice during the layer formation to impose through the substrate an applied force condition opposing or enhancing the retention of stress (e.g., compressive or tensile force) in the product layer. The method of the invention has particular importance for relatively thick (up to 100 microns) biocompatible metals such as platinum, iridium and titanium used as interconnections; iridium oxide and titanium nitride electrodes as well as various dielectric films used for biomedical encapsulation.

This method is also applicable in the converse to the production of layers that have tensile growth stress. In such case, the thermal expansion mismatch strain can be compensated in the practice of the invention by heating the substrate at the deposition temperature. In this way, there is little or no stress during deposition, and a stress is created during cooling, but the stress is then relieved by removing the wafer constraint.

In certain embodiments, the substrate surface has secured thereto a member formed of a material having a different coefficient of thermal expansion from the substrate, and wherein the formation of the product film of the film-forming material comprises heating and/or cooling of the substrate and member secured thereto.

Depending on the particular embodiment, the substrate surface may be smooth or irregular, where when the substrate surface is irregular in may have holes or trenches or analogous structures that are to be filled with the deposited material.

In certain embodiments, deposition conditions (e.g., gas makeup, power) may be selected which yield a porous coating. For example, the pressure of the reactive gases may be chosen to provide for a desired porosity in the final product. For example, where N₂ is the reactive gas, pressures ranging from 0.01 to 760 torr, such as 0.1 to 100 torr, are employed to produce a porous structure of many metals, such as platinum, gold, ruthenium, indium and molybdenum. Where C₂H₈ is the reactive gas, pressures ranging from 0.01 to 760 torr, such as 0.1 to 100 torr, are employed to produce a porous structure of many metals, such as platinum, gold, ruthenium, iridium and molybdenum. Further details regarding deposition conditions of interest are provided in copending PCT. Application serial no. PCT/US2007/______ titled: “Metal Binary and Ternary Compounds Produced by Cathodic Arc Deposition,” filed on even date herewith, the disclosure of which is herein incorporated by reference.

In certain embodiments, one or more masks may be employed in conjunction with the cathodic arc deposition protocol. Such masks may provide for any desirable shape of deposited structured. Any convenient mask, such as conventional masks employed in photolithographic processing protocols, etc., may be employed.

As described above, the structure that is deposited by the subject methods may have a variety of different configurations, and may be a layer, a lead, have a three-dimensional configuration, etc., depending on the intended function of the deposited structured.

The composition of the deposited structure may be selected based on the choice of cathode material and atmosphere of plasma generation. As such, a particular cathode material and atmosphere of plasma generation are selected to produce a metallic layer of desired composition. In certain embodiments, the cathode is made up of a metal or metal alloy, where metals of interest include, but are not limited to: gold (au), silver (ag), nickel (ni), osmium (os), palladium (pd), platinum (pt), rhodium (rh), iridium (ir) titanium (ti), and the like.

The ion beam may be produced in a vacuum in those embodiments where the deposited structure is to have the same composition as the cathode. In yet other embodiments where the deposited structure is to be an alloy of a metal with another element, such as a carbon, oxygen or nitrogen, the plasma may be produced in an atmosphere of the other element, e.g., an oxygen containing atmosphere, a nitrogen containing atmosphere, a carbon containing atmosphere, etc.

In certain embodiments, a gradient of a second element in the cathode material is produced in the deposited structure, e.g., by modifying the atmosphere while the plasma is being generated, such that the amount of the second element in the atmosphere is changed, e.g., increased or decreased, while deposition is occurring.

In certain embodiments, the ion beam that is contacted with the substrate surface is unfiltered, such that the ion beam includes macroparticles of the cathode material. In yet other embodiments, the ion beam may be filtered such that the beam is substantially if not completely free of macroparticles is contacted with the substrate surface. Any convenient filtration protocol may be employed, such as those described in U.S. Pat. Nos. 6,663,755; 6,031,239; 6,027,619; 5,902,462; 5,317,235 and 5,279,723 and published U.S. Application Nos. 20050249983; 20050181238; 20040168637; 20040103845 and 20020007796; the disclosures of which are herein incorporated by reference.

As reviewed above, in certain embodiments, the cathodic arc deposited structure is a conductive element that conductively joins two or more structures of an implantable medical device, e.g., a conductive feedthrough or via as shown in FIGS. 6A and 6B. In certain of these embodiments, a multi-layered biocompatible structure intended for use as an implant in a human body is fabricated in which a microprocessor or other component is configured in different layers and interconnected vertically through insulating layers which separate each circuit layer of the structure, where the vertical interconnection is produced via cathodic arc deposition as described herein. Each circuit layer can be fabricated in a separate wafer or thin film material and then transferred onto the layered structure and interconnected as described below.

A biocompatible layer metal conductor, e.g., made up of Pt, Ir, Ti, or alloys thereof, is deposited on the patterned silicon substrate via cathodic arc deposition techniques, e.g., through an external (e.g., silicon) mask to a define three-dimensional electrical circuit and an electrical connection through vias formed in the silicon substrate or case containing a microprocessor or other component. These methods include exposing the first portion to a beam of substantially fully ionized metallic ions like, e.g., as produced above. The method uses unfiltered as well filtered Cathodic Vacuum Arc techniques to generate the highly directional ion beam and permits the formation of a conformal metal coating, even in high aspect ratio vias and trenches. The method also permits the in-filling of vias and trenches to form conductive interconnects, e.g., deposition of platinum thin and thick films and interconnections

In certain embodiments, the structures are vertically stacked and interconnected circuit elements for data processing, control systems, and programmable computing for use in implantable devices. In certain embodiments, the structures include interconnecting circuitry and microprocessors which are fabricated in the same or separate semiconductor wafers and then stacked. This circuitry may include a number of thin film metal wires that are normally routed along the surface of silicon or other suitable material. In the present invention the functional blocks of the circuit may be divided into two or more vertically arranged sections with one section of the circuit on a bulk chip and the remaining blocks, like SI based wafer with cavities which contain an embedded microprocessor chip and components, being electrically connected through an intervening vias produced via the cathodic arc deposition protocols described herein.

Circuits can be formed in bulk silicon, silicon oxide, or in III-V materials such as gallium arsenide, or in composite structures including bulk Si, SOI, and/or thin film GaAs. The various layers of the device can be stacked using an insulating layer that bonds the layers together and conductive interconnects or vertical busses extending through the insulating layer which may include a polymeric material such as an adhesive. Thermal and electrical shielding can be employed between adjacent circuit layers to reduce or prevent thermal degradation or cross-talk.

Wire bond pads on the bulk chip or on the thin film layers of the structure may be present for communicating with the package, e.g., where the chips are placed in a leadless chip carrier. These pads need to be large enough that wires can be bonded to them. Interconnection pads are used to connect the different layers of the circuit together. These pads can be considerably smaller than traditional wire bond pads because the methods of interconnection employ cathodic arc metal deposition.

Accordingly, embodiments of the invention include methods of fabricating an implantable active electronic device which includes a data processor, where the methods include forming a first metal, e.g., Pt, based electrical circuit on a first layer of semiconductor material, e.g., a bulk semiconductor wafer (Si, SiC, GaAs, InP, etc., or a wafer of a dielectric material (e.g., TiO₂ Al₂O₃, AlN, SiO₂) etc; forming a second circuit of the data processor in a second layer of semiconductor material; and electrically interconnecting the second layer to the first layer with a cathodic arc deposited metal, e.g., Pt, conductor via depositing by cathodic arc deposition up to 100 micron deep vias connecting the first processor circuit with the second embedded processor circuit with an interconnect extending between the two circuits so that data processor signals can be conducted between the first data processor circuit and the second data processor circuit. As desired, the methods may further include producing a plurality of additional circuit layers over the first and second circuit layers.

In certain embodiments, methods of fabricating a data processor include: forming a first circuit of an implant in a first layer of semiconductor or dielectric material; forming a second circuit of a data processor in a second layer of semiconductor material; bonding the second layer to the first layer with a bonding layer; and applying a Pt, Ti or other biocompatible metallization layer via cathodic arc deposition for electrically connecting the first circuit and the second circuit, the metallization layer flowing from the second layer through the hole to the first layer

### Cathodic Arc Deposition Systems

Also provided are cathodic arc deposition systems that may be employed in practicing the subject methods to make implantable medical devices that include cathodic arc produced structures. Embodiments of the subject systems include a cathodic arc plasma source and a substrate mount. The cathodic arc plasma source (i.e., plasma generator) may vary, but in certain embodiments includes a cathode, one or more anodes and a power source between the cathode and anode(s) for producing an electrical arc sufficient to produce ionized cathode material from the cathode during plasma generation. The plasma generator may generate a DC or pulsing plasma beam, including positively charged ions from a cathode target.

The substrate mount is configured for holding a substrate on which a structure is to be deposited. In certain embodiments, the substrate mount is one that includes a temperature modulator for controlling the temperature of a substrate present on the mount, e.g., for increasing or decreasing the temperature of a substrate on the mount to a desired value. Any convenient temperature modulator may be operatively connected to the mount, such as a cooling element, heating element etc. In certain embodiments, a temperature sensor may be present for determining the temperature of a substrate present on the mount.

In certain embodiments, the system is configured so that the distance between the substrate mount and the cathode may be adjusted. In other words, the system is configured such that the substrate mount and cathode may be moved relative to each other. In certain embodiments, the system is configured so that the substrate mount can be moved relative to the cathode so that the distance between the two can be increased or decreased as desired. In certain embodiments, the system is configured so that the cathode can be moved relative to the substrate mount so that the distance between the two can be increased or decreased as desired. As desired, the system may include an element for determining the proper distance to position the substrate mount and cathode relative to each other in view of one or more input parameters, e.g., cathode material, energy, substrate specifics, deposition atmosphere, to produce a thick, stress-free product layer, e.g., by ensuring that any compressive forces present in the deposited material are canceled by tensile forces of the substrate, as reviewed above.

The cathodic arc plasma generation element and substrate are, in certain embodiments, present in a sealed chamber which provides for the controlled environment, e.g., a vacuum or controlled atmosphere, where the two components of the system may be present in the same chamber or different chambers connected to each other by an ion conveyance structure which provides for movement of the ions from the cathode to, the substrate.

In certain embodiments, the system further includes a fitter component which serves to filter macroparticles from the produced plasma so that a substantially if not completely macro-particle free ion beam contacts the substrate. Any convenient filtering component may be present, where filtering components of interest include, but are not limited to: those described in U.S. Pat. Nos. 6,663,755; 6,031,239; 6,027,619; 5,902,462; 5,317,235 and 5,279,723 and published U.S. Application Nos. 20050249983; 20050181238; 20040168637; 20040103845 and 20020007796; the disclosures of which are herein incorporated by reference. In certain embodiments, the filter element has two bends such that there is no line of sight and no single bounce path through the filter between the source and the substrate. In certain embodiments, the system further includes a beam steering arrangement, which steers the plasma beam through a filter and onto the substrate.

In certain embodiments, the system includes an ion beam modulator, e.g., a beam biasing arrangement for applying a pulsed, amplitude modulated electrical bias to a filtered plasma beam. In these embodiments, the biasing arrangement comprises a processing device and a pulse generator module, the pulse generator module generating the pulsed, amplitude modulated electrical bias under the control of the processing device in which the pulse generator module includes a programmable logic device, a power supply and a switching circuit, the switching circuit being controlled by the programmable logic device and an output of the power supply being coupled to the substrate via the switching circuit, wherein the programmable logic device controls the operation of both the power supply and the switching circuit.

In certain embodiments, the system further includes an element for biasing the substrate. In certain of these embodiments, the biasing operates both to dissipate electrostatic charge accruing on the substrate due to the deposition of positive ions and to ensure that the energy of incident ions falls in a predetermined energy range.

Cathodic arc deposition systems are further described in U.S. Provisional Application Ser. No. 60/805,576 titled “Implantable Medical Devices Comprising Cathodic Arc Produced Structures,” and filed on Jun. 22, 2006; the disclosure of which is herein incorporated by reference.

### Systems

Also provided are systems that include one more implantable medical devices that include a cathodic arc produced component according to the invention.

For example, systems that include an implantable device having a cathodic arc produced antenna, such as a patch antenna, e.g., as described above, are provided. Such systems of the invention may be viewed as systems for communicating information within the body of subject, e.g., human, where the systems include both a first implantable medical device comprising a transceiver configured to transmit and/or receive a signal; and a second device comprising a transceiver configured to transmit and/or receive a signal, wherein at least one of the first and second devices includes a microstrip antenna according to the invention, e.g., as described above.

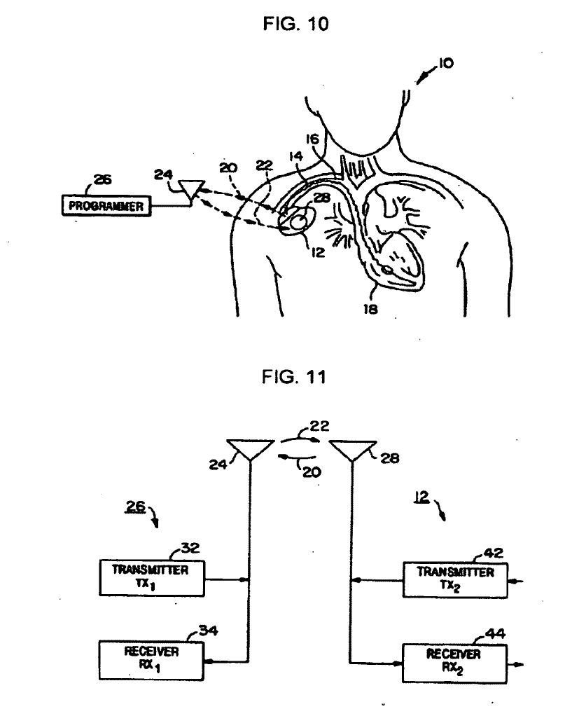

One embodiment of a system of the invention is shown in FIG. 10, where the system includes an implantable medical device, e.g., an IPG, and an external programming unit. FIG. 10 is a simplified schematic diagram of bi-directional telemetry communication between an external programmer 26 and an implanted medical device, e.g., a cardiac pacemaker IPG 12, in accordance with the present invention. The IPG 12 is implanted in the patient 10 beneath the patient's skin or muscle and is typically oriented to the skin surface. IPG 12 is electrically coupled to the heart 18 of the patient 10 through pace/sense electrodes and lead conductor(s) of at least one cardiac pacing lead 14. The IPG 12 contains an operating system that may employ a microcomputer or a digital state machine for timing sensing and pacing functions in accordance with a programmed operating mode and a power source. The IPG 12 also contains sense amplifiers for detecting cardiac signals, patient activity sensors or other physiologic sensors for sensing the need for cardiac output, and pulse generating output circuits for delivering pacing pulses to at least one heart chamber of the heart 18 under control of the operating system in a manner well known in the prior art. The operating system includes memory registers or RAM for storing a variety of programmed-in operating mode and parameter values that are used by the operating system. The memory registers or RAM may also be used for storing data compiled from sensed cardiac activity and/or relating to device operating history or sensed physiologic parameters for telemetry out on receipt of a retrieval or interrogation instruction. All of these functions and operations are well known in the art, and many are employed in other programmable, implantable medical devices to store operating commands and data for controlling device operation and for later retrieval to diagnose device function or patient condition.

Programming commands or data are transmitted between an IPG RF telemetry antenna 28 within or on a surface of the IPG 12 and an external RF telemetry antenna 24 associated with the external programmer 26. The external RF telemetry antenna 24 can be located on the cape of the external programmer some distance away from the patient 10. For example, the external programmer 26 and external RF telemetry antenna 24 may be on a stand a few meters or so away from the patient 10. Moreover, the patient may be active and could be exercising on a treadmill or the like during an uplink telemetry interrogation of real time ECG or physiologic parameters. The programmer 26 may also be designed to universally program existing IPGs that employ the conventional ferrite core, wire coil, RF telemetry antenna of the prior art and therefore also have a conventional programmer RF head and associated software for selective use with such IPGs.

In an uplink telemetry transmission 20, the external RF telemetry antenna 24 operates as a telemetry receiver antenna, and the IPG RF telemetry antenna 28 operates as a telemetry transmitter antenna. Conversely, in a downlink telemetry transmission 30, the external RF telemetry antenna 24 operates as a telemetry transmitter antenna; and the IPG RF telemetry antenna 28 operates as a telemetry receiver antenna.

Turning to FIG. 11, it is a simplified circuit block diagram of major functional telemetry transmission blocks of the external programmer 26 and IPG 12 of FIG. 10. The external RF telemetry antenna 24 within the programmer 26 is coupled to a telemetry transceiver comprising a telemetry transmitter 32 and telemetry receiver 34. The telemetry transmitter 32 and telemetry receiver 34 are coupled to control circuitry and registers operated under the control of a microcomputer and software as described in the above-incorporated, commonly assigned, patents and pending applications. Similarly, within the IPG 12, the IPG RF telemetry antenna 28 is coupled to a telemetry transceiver comprising a telemetry transmitter 42 and telemetry receiver 44. The telemetry transmitter 42 and telemetry receiver 44 are coupled to control circuitry and registers operated under the control of a microcomputer and software as described in the above-incorporated, commonly assigned, patents and pending applications.

In an uplink telemetry transmission 20, the telemetered data may be encoded in any convenient telemetry formats. For example, the data encoding or modulation may be in the form of frequency shift key (FSK) or differential phase shift key (DPSK) modulation of the carrier frequency, for example. To initiate an uplink telemetry transmission 20, the telemetry transmitter 32 in external programmer 26 is enabled in response to a user initiated INTERROGATE command to generate an INTERROGATE command in a downlink telemetry transmission 22. The INTERROGATE command is received and demodulated in receiver 44 and applied to an input of the implantable medical device central processing unit (CPU), e.g. a microcomputer (not shown). The implantable medical device microcomputer responds by generating an appropriate uplink data signal that is applied to the transmitter 42 to generate the encoded uplink telemetry signal 20. Any of the above described data encoding and transmission formats may be employed.

The system of FIGS. 10 and 11 described above is merely illustrative and only one type of system in which the subject antennas may be employed. The systems may have a number of different components or elements, where such elements may include, but are not limited to: sensors; effectors; processing elements, e.g., for controlling timing of cardiac stimulation, e.g., in response to a signal from one or more sensors; telemetric transmitters, e.g., for telemetrically exchanging information between the implantable medical device and a location outside the body; drug delivery elements, etc.

In certain embodiments, the implantable medical systems are ones that are employed for cardiovascular applications, e.g., pacing applications, cardiac resynchronization therapy applications, etc.

Use of the systems may include visualization of data obtained with the devices. Some of the present inventors have developed a variety of display and software tools to coordinate multiple sources of sensor information which will be gathered by use of the inventive systems. Examples of these can be seen in international PCT application serial no. PCT/US2006/012246; the disclosure of which application, as well as the priority applications thereof are incorporated in their entirety by reference herein.

Data obtained using the implantable embodiments in accordance with the invention, as desired, can be recorded by an implantable computer. Such data can be periodically uploaded to computer systems and computer networks, including the Internet, for automated or manual analysis.

Uplink and downlink telemetry capabilities may be provided in a given implantable system to enable communication with either a remotely located external medical device or a more proximal medical device on the patient's body or another multi-chamber monitor/therapy delivery system in the patient's body. The stored physiologic data of the types described above as well as real-time generated physiologic data and non-physiologic data can be transmitted by uplink RF telemetry from the system to the external programmer or other remote medical device in response to a downlink telemetry transmitted interrogation command. The real-time physiologic data typically includes real time sampled signal levels, e.g., intracardiac electrocardiogram amplitude values, and sensor output signals including dimension signals developed in accordance with the invention. The non-physiologic patient data includes currently programmed device operating modes and parameter values, battery condition, device ID, patient ID, implantation dates, device programming history, real time event markers, and the like. In the context of implantable pacemakers and ICDs, such patient data includes programmed sense amplifier sensitivity, pacing or cardioversion pulse amplitude, energy, and pulse width, pacing or cardioversion lead impedance, and accumulated statistics related to device performance, e.g., data related to detected arrhythmia episodes and applied therapies. The multi-chamber monitor/therapy delivery system thus develops a variety of such real-time or stored, physiologic or non-physiologic, data, and such developed data is collectively referred to herein as “patient data”.

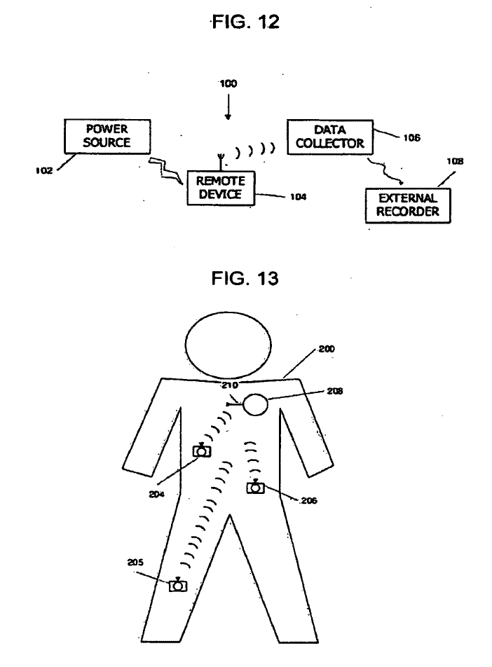

FIG. 12 is a block diagram of a medical diagnostic and/or treatment system 100 according to another embodiment of the present invention. Platform 100 includes a power source 102, a remote device 104, a data collector 106, and an external recorder 108. In operation, remote device 104 is placed inside a patient's body (e.g., ingested or implanted) and receives power from power source 102, which may be located inside or outside the patient's body.

Remote device 104, is an electronic, mechanical, or electromechanical device that may include any combination of sensor, effector and/or transmitter units. A sensor unit detects and measures various parameters related to the physiological state of a patient in whom remote device 104 is implanted. An effector unit performs an action affecting some aspect of the patient's body or physiological processes under control of a sensor unit in the remote device or an external controller. A transmitter unit transmits signals, including, e.g., measurement data from a sensor unit or other signals indicating effector activity or merely presence of the remote device, to data collector 106. In certain embodiments, transmission is performed wirelessly.

Power source 102, can include any source of electrical power that can be delivered to remote device 104. In some embodiments, power source 102 may be a battery or similar self-contained power source incorporated into remote device 104. In other embodiments, power source 102 is external to the patient's body and delivers power wirelessly.

Data collector 106 may be implanted in the patient or external and connected to the patient's skin. Data collector 106 includes a receiver antenna that detects signals from a transmitter unit in remote device 104 and control logic configured to store, process, and/or retransmit the received information. In embodiments where remote device 104 does not include a transmitter, data collector 106 may be omitted.

External recorder 108 may be implemented using any device that makes the collected data and related information (e.g., results of processing activity in data collector 106) accessible to a practitioner. In some embodiments, data collector 106 includes an external component that can be read directly by a patient or health care practitioner or communicably connected to a computer that reads the stored data, and that external component serves as external recorder 108. In other embodiments, external recorder 108 may be a device such as a conventional pacemaker wand that communicates with an internal pacemaker can or other data collector, e.g., using RF coupling in the 405-MHz band.

Platform 100 can include any number of power sources 102 and remote devices 104, which may be viewed as implantable medical devices. In some embodiments, a sensor/effector network (system) can be produced within the patient's body to perform various diagnostic and/or treatment activities for the patient. For instance, FIG. 13 shows a patient 200 with multiple remote devices 204, 205, 206 implanted at various locations in his (or her) body. Remote devices 204, 205, 206 might be multiple instances of the same device, allowing local variations in a parameter to be measured and/or various actions to be performed locally. Alternatively, remote devices 204, 205, 206 might be different devices including any combination of sensors, effectors, and transmitters. In certain embodiments, each device is configured to at least one of: (i)

transmit a signal via a quasi electrostatic coupling to the body of the patient; and (ii) receive the transmitted signal via a quasi electrostatic coupling to the body of the patient. The number of remote devices in a given system may vary, and may be 2 or more, 3 or more, 5 or more, about 10 or more, about 25 or more, about 50 or more, etc. A data collector 208 is equipped with an antenna 210 and detects the signals transmitted by remote devices 204, 205, 206. Since the remote devices advantageously transmit signals wirelessly, applications of the platform are not limited by the difficulty of running wires through a patient's body. Instead, as will become apparent, the number and placement of remote devices in a patient's body is limited only by the ability to produce devices on a scale that can be implanted in a desired location.

The description of the present invention is provided herein in certain instances with reference to a patient. As used herein, the term “patient” refers to a living entity such as an animal. In certain embodiments; the animals are “mammals” or “mammalian,” where these terms are used broadly to describe organisms which are within the class mammalia, including the orders carnivore (e.g., dogs and cats), rodentia (e.g., mice, guinea pigs, and rats), lagomorpha (e.g. rabbits) and primates (e.g., humans, chimpanzees, and monkeys). In certain embodiments, the subjects, e.g., patients, are humans.

Also provided are methods of using the systems of the invention. The methods of the invention generally include: providing a system of the invention, e.g., as described above, that includes first and second medical devices, one of which may be implantable; and transmitting a signal between the first and second devices of the system via a microstrip antenna present on at least one of the devices. The provides may include implanting at least the first medical device into a subject, depending on the particular system being employed. In certain embodiments, the transmitting step includes sending a signal from the first to said second device. In certain embodiments, the transmitting step includes sending a signal from the second device to said first device. The signal may transmitted in any convenient frequency, wherein certain embodiments the frequency ranges from about 400 to about 405 MHz. The nature of the signal may vary greatly, and may include one or more data obtained from the patient, data obtained from the implanted device on device function, control information for the implanted device, power, etc.

### Kits

Also provided are kits that include the implantable medical devices, such as an implantable pulse generator, e.g., as reviewed above. For example, the kits may include a device, e.g., either implantable or ingestible, that includes a patch antenna of the invention, e.g., as described above. In certain embodiments, the kits may include two or more such devices. In certain embodiments, the kits further include at least one additional component, e.g., an implantation device (such as tool, guidewire, etc.), a receiver, etc.

In certain embodiments of the subject kits, the kits will further include instructions for using the subject devices or elements for obtaining the same (e.g., a website URL directing the user to a webpage, which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. An implantable medical device comprising a cathodic arc produced structure.

2. The implant according to claim 1, wherein said cathodic arc produced structure is a stress-free structure having a thickness ranging from about 1 μm to about 100 μm.

3. The implant according to claim 2, wherein said structure is a layer that covers at least a portion of surface a component of said implant.

4. The implant according to claim 3, wherein said layer seals an internal volume of said implant.

5. The implant according to claim 2, wherein said structure is a component of said implant.

6. The implant according to claim 5, wherein said component is a conductive element.

7. The implant according to claim 6, wherein said conductive element is present in a high aspect ratio passage of said implant.

8. The implant according to claim 5, wherein said component is an effector.

9. The implant according to claim 8, wherein said effector is an actuator.

10. The implant according to claim 8; wherein said effector is a sensor.

11. The implant according to claim 1, wherein said cathodic arc produced structure is a microstrip antenna.

12. The medical device according to claim 11, wherein said microstrip antenna comprises a cathodic arc produced radiator patch layer on a surface of a substrate layer.

13. The implant according to claim 1, wherein said cathodic arc produced structure is a crenulated layer.

14. The implant according to claim 13, wherein said crenulated layer is present on an implant.

15. The implant according to claim 14, wherein said crenulated layer further comprises a pharmaceutically active agent.

16. The implant according to claim 1, wherein said cathodic arc produced structure is a porous layer.

17. The implant according to claim 16, wherein said porous layer is part of a high surface area electrode.

18. A method of producing a metallic structure on a substrate, said method comprising:
contacting a cathodic arc generated metallic ion plasma with a surface of said substrate to produce a deposited metallic stress-free structure having a thickness of about 1 μm or greater on said substrate.

19. The method according to claim 18, wherein said contacting occurs in a manner such that compressive and tensile forces experienced by said deposited metal structure substantially cancel each other out so that said deposited metal structure is stress-free.

20. The method according to claim 18, wherein plasma is contacted with said surface in a direction that is substantially orthogonal to a plane of said surface.

21. The method according to claim 18, wherein said method is a method of producing a portion of an implantable medical device.

22. The method according to claim 21, wherein said portion is a component of said implantable medical device.

23. The method according to claim 22, wherein said component is a conductive element.

24. The method according to claim 23, wherein said conductive element is present in a high aspect ratio passage of said implant.

25. The method according to claim 24, wherein said high aspect ratio structure has a height to width ratio ranging from about 1 to about 50.

26. The method according to claim 21, wherein said portion is a metallic layer that covers at least a portion of said surface.

27. The method according to claim 26, wherein said metallic layer seals an internal space of said substrate.

28. The method according to claim 18, wherein said metallic structure comprises a physiologically compatible metal.

29. The method according to claim 28, wherein said metal is chosen from platinum, iridium and titanium.

30. The method according to claim 18, wherein said plasma is generated in a vacuum.

31. The method according to claim 18, wherein said plasma is generated in the presence of oxygen.

32. The method according to claim 18, wherein said plasma is generated in the presence of nitrogen.

33. The method according to claim 18, wherein said plasma is generated in the presence of carbon.

34. A cathodic arc plasma deposition system comprising:
a cathodic arc plasma source; and
a substrate mount, wherein said substrate mount includes a temperature modulator for modulating the temperature of a substrate mounted thereon.

35. The cathodic arc plasma deposition system according to claim 34, wherein the distance between said substrate mount and said cathodic arc plasma source may be adjusted.

36. The cathodic arc plasma deposition system according to claim 35, wherein said system includes a plasma filter.

37. The cathodic arc plasma deposition system according to claim 35, wherein said system includes a plasma beam biasing element.
